(19) United States
(12) Patent Application Publication
(10) Pub. No.: US 2025/0271388 A1
(43) Pub. Date: Aug. 28, 2025

(54) ZnO NANORODS PROVIDED WITH ORTHOGONAL OXIDIZED COPPER NANOPLATES AND CORRESPONDING GAS SENSOR

(71) Applicant: Luxembourg Institute of Science and Technology (LIST), Esch-sur-Alzette (LU)

(72) Inventors: Renaud Leturcq, Luxembourg (LU); Jean-Sebastien Thomann, Gorcy (FR); Rutuja Bhusari, Esch-sur-Alzette (LU)

(21) Appl. No.: 18/858,471

(22) PCT Filed: Apr. 20, 2023
(86) PCT No.: PCT/EP2023/060303

§ 371 Date: Oct. 21, 2024

(30) Foreign Application Priority Data
Apr. 21, 2022 (LU) LU501895

(51) Int. Cl. G01N 27/30

(52) U.S. Cl. CPC G01N 27/30

(57) ABSTRACT

a metal oxide material comprising ZnO nanorods and oxidized copper nanoplates extending transversally to the ZnO nanorod; a method for synthetizing the metal oxide material; and a gas sensor comprising a substrate; at least two electrodes deposited on the substrate; a gas sensing layer comprising ZnO nanorods deposited on the at least two electrodes and on the substrate between said at least two electrodes, said gas sensing layer showing an electrical resistivity that varies when contacted by the gas; wherein the ZnO nanorods are provided with transversal nanoplates of CuO so as to confer sensitivity of the gas sensing layer to the gas being at least one of O₂, H₂, CO, ethanol and NO₂.

### CROSS-REFERENCE TO RELATED APPLICATIONS

The present invention is the US national stage under 35 U.S.C. § 371 of International Application No. PCT/EP2023/060303 which was filed on Apr. 20, 2023, and which claims the priority of application LU501895 filed on Apr. 21, 2022, the contents of which (text, drawings and claims) are incorporated here by reference in their entirety.

### FIELD

The invention is directed to the field of metal oxide heterostructures for various applications including gas sensing, photocatalysis and water splitting.

### BACKGROUND

Prior art patent document published CN 106814113 A discloses a semiconductor oxide gas sensor based on ZnO nanorods onto which CuO particles are grown, so as to provide a good sensitivity to H₂S. The CuO particles are uniformly distributed on the ZnO nanorods and show a diameter comprised between 20 and 50 nm. The resulting gas sensor shows a good sensitivity to H₂S and also a good selectivity compared with other gases like SO₂, Cl₂ and O₂, in particular at 50° C.

Prior art patent document published CN 107537501 A discloses a method for producing ZnO/CuO composite material intended to be used for gas sensing, photocatalysis and water splitting. The ZnO/CuO composite material comprises a flower-like hierarchical structure of ZnO matrix and CuO nanoparticles attached to the surface of said matrix. The size of the CuO nanoparticles is comprised between 10 and 50 nm whereas the size of the hierarchical structure of ZnO is comprised between 10 and 20 μm.

Rutuja Bhusari, Jean-Sebastien Thomann, Jérême Guillot, Renaud Leturcq, “Morphology control of copper hydroxide based nanostructures in liquid phase synthesis”, Journal of Crystal Growth, Volume 570, 2021, 126225, ISSN 0022-0248, https://doi.org/10.1016/j.jcrysgro.2021.126225 (https://www.sciencedirect.com/science/article/pii/S0022024821002001), discloses synthesis and study of copper hydroxide based nanostructures as templates for formation of CuO, more particularly the template-free bottom-up synthesis and shape control of copper hydroxide based nanostructures grown in liquid phase, from 1D nanowires to 2D layered nanoplatelets and 3D nanocrystals.

The gas sensitivity of the above described ZnO/CuO composite materials, remains limited, in particular at room temperature or controlled temperatures below 200° C. It is therefore desirable to increase the gas sensitivity and/or selectivity of these materials.

### SUMMARY

The invention has for technical problem to overcome at least one drawback of the above cited prior art. More specifically, the invention has for technical problem to provide a metal oxide heterostructure with an increased chemiresistive gas sensitivity and/or selectivity.

The invention is directed to a gas sensor comprising a substrate; at least two electrodes deposited on the substrate; a gas sensing layer comprising ZnO nanorods deposited on the at least two electrodes and on the substrate between said at least two electrodes, said gas sensing layer showing an electrical resistivity that varies when contacted by the gas; wherein the ZnO nanorods are provided with transversal nanoplates of oxidized copper so as to confer sensitivity of the gas sensing layer to the gas being at least one of O₂, H₂, CO, ethanol and NO₂.

The oxidized copper forming the transversal nanoplates comprises CuO. It can also comprise Cu₂(OH)₃Cl and Cu(OH)₂.

According to an exemplary embodiment, the ZnO nanorods have a length comprised between 1 and 10 μm.

According to an exemplary embodiment, the ZnO nanorods have a diameter comprised between 50 and 600 nm.

According to an exemplary embodiment, the ZnO nanorods extend along a longitudinal axis and the transversal oxidized copper nanoplates are perpendicular to the longitudinal axis within a tolerance of ±15°, in various instances within a tolerance of ±10°.

According to an exemplary embodiment, the ZnO nanorods provided with transversal nanoplates of oxidized copper have a number of the transversal nanoplates of CuO that is comprised between 10 and 100 per μm of length of ZnO nanorod.

According to an exemplary embodiment, the ZnO nanorods provided with transversal nanoplates of oxidized copper are obtainable by the method of any one of claims 13-18.

Advantageously, the gas sensor further comprises an electrical resistor dimension for bringing the substrate, the at least two electrodes and the gas sensing layer at a temperature of at least 100° C. and up to 250° C.

The invention is also directed to a metal oxide material comprising ZnO nanorods; and oxidized copper particles attached to the ZnO nanorods; wherein the oxidized copper particles are nanoplates extending transversally to the ZnO nanorods.

According to an exemplary embodiment, the ZnO nanorods have a length comprised between 1 and 10 μm.

According to an exemplary embodiment, the ZnO nanorods have a diameter comprised between 50 and 600 nm.

According to an exemplary embodiment, the ZnO nanorods extend along a longitudinal axis and the transversal oxidized copper nanoplates are perpendicular to the longitudinal axis within a tolerance of ±15°, in various instances within a tolerance of ±10°.

According to an exemplary embodiment, the ZnO nanorods provided with transversal nanoplates of oxidized copper have a number of the transversal nanoplates of CuO that is comprised between 10 and 100 per μm of length of ZnO nanorod.

According to an exemplary embodiment, the ZnO nanorods provided with transversal nanoplates of oxidized copper are obtainable by the method of any one of claims 13-18.

The invention is also directed to a method of synthetizing ZnO nanorods provided with Cu-based nanoplates, comprising the following successive steps: (a) preparing ZnO nanorods; (b) mixing the ZnO nanorods with a Cu precursor in a solution; (c) increasing the pH of the solution, so as to form Cu(OH)₂ nanowires in the solution; (d) lowering the pH of the solution, so as to form Cu₂(OH)₃Cl nanoplates on the ZnO nanorods.

Advantageously, the solution is water based.

According to an exemplary embodiment, the method comprises the further step: (e) annealing the ZnO nanorods provided with the Cu₂(OH)₃Cl nanoplates so as to transform at least partially the Cu₂(OH)₃Cl nanoplates into CuO nanoplates.

According to an exemplary embodiment, annealing at step (e) is achieved at a temperature of at least 100° C. during at least one hour, in various instances at least two hours.

According to an exemplary embodiment, annealing at step (e) is achieved at a temperature of at least 200° C. during at least one hour, in various instances at least two hours.

According to an exemplary embodiment, step (c) comprises adding NaOH so as to increase the pH to at least 12.

According to an exemplary embodiment, step (d) comprises adding HCl so as to lower the pH to a value comprised between 6 and 8.

Advantageously, the Cu precursor comprises CuCl₂.

The invention is particularly interesting in that it provides a metal oxide material and a corresponding gas sensor showing an improved chemiresistive sensitivity in the presence of different gases, essentially by increasing the total contact surface with the gas per unit of metal oxide material.

The range of applications of the metal oxide material of the invention is broad and therefore is not limited to gas sensing and, in the context of gas sensing, is not limited to sensing a unique or specific type of gas. the working principle of chemiresistive gas sensor is the change in resistance due to chemical redox reactions and is based on the Ohm's law, V=R·I. When a target gas interacts with the oxygen adsorbed or with the elements on the surface of a chemiresistive sensor, there occurs a reaction. This leads to change in the resistance of the material. The ability of the metal oxide surface to react with the target gas depends on the properties of the surface and the oxygen species adsorbed on the surface. The adsorption of oxygen at the surface changes the resistance of the semiconductor material formed by the metal oxide.

### DRAWINGS

FIG. 1 illustrates the heterostructure of ZnO nanorods with transversal nanoplates of CuO, according to the invention.

FIG. 2 illustrates the synthesis of the ZnO nanorods with transversal nanoplates of Cu₂(OH)₃Cl, according to the invention.

FIG. 3 illustrates the geometry of nanoparticles versus nanoplates.

FIG. 4 illustrates a gas sensor according to two variants, according to the invention.

FIG. 5 is a graphic illustrating the electrical behavior of a gas sensor according to the invention in contact with various gases.

### DETAILED DESCRIPTION

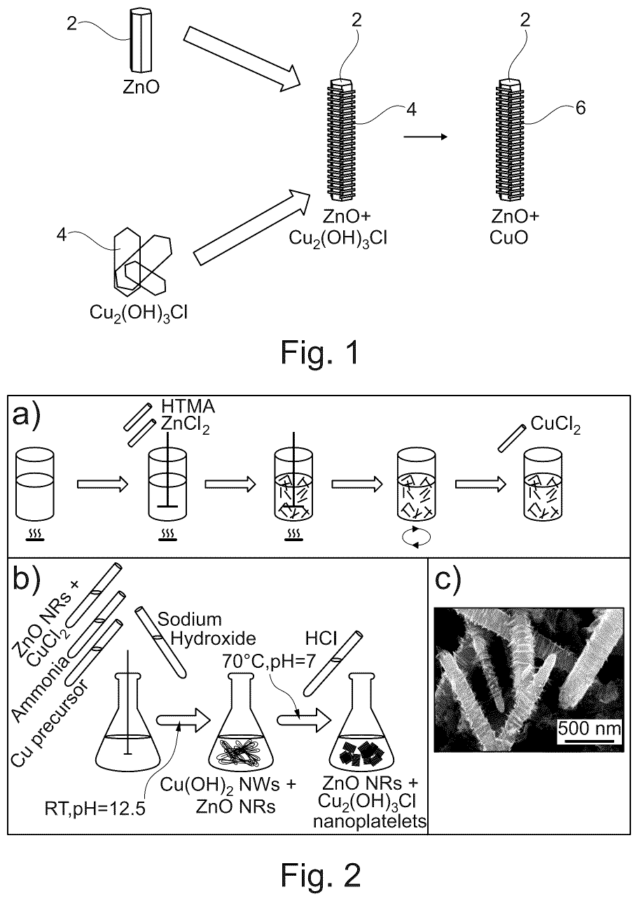

FIG. 1 illustrates the heterostructure of ZnO nanorods provided with transversal nanoplates of CuO, according to the invention.

As this is apparent, ZnO nanorods 2 are provided and Cu₂(OH)₃Cl nanoplates 4 that are growth on the ZnO nanorods 2. The ZnO nanorods 2 with the Cu₂(OH)₃Cl nanoplates 4 are then annealed resulting in a conversion of the Cu₂(OH)₃Cl nanoplates 4 into CuO nanoplates 6.

As this is apparent, the nanoplates of Cu₂(OH)₃Cl nanoplates 4 and more particularly the CuO nanoplates 6 are perpendicular to the longitudinal axis of the ZnO nanorod 2 and are distributed evenly along and around the nanorod 2. This particular arrangement of CuO nanoplates on ZnO nanorods is particularly advantageous in that it maximizes the effective surface of the copper oxide nanoplates per nanorod and thereby increases the catalytic effect useful for the various applications including gas sensing, photocatalysis and water splitting.

FIG. 2 illustrates a method for synthetizing the above ZnO nanorods 2 with the Cu₂(OH)₃Cl nanoplates 4.

More specifically, section a) of FIG. 2 illustrate the synthetizing of ZnO nanorods, comprising essentially mixing ZnCl₂ with Hexamethylenetetramine (HTMA), also known as methenamine, hexamine, or urotropin, and being heterocyclic organic compound with the formula (CH₂)₆N₄, with water, heating and mixing, and centrifugating. More specifically, the method comprises the following steps:
as illustrated in section a)-1 of FIG. 2, providing a solution, i.e., water, and heating said solution, i.e., to a temperature comprised between 5° and 100° C., in various instances comprised between 6° and 80° C.;
as illustrated in section a)-2 of FIG. 2, adding and dissolving ZnCl₂ and Hexamethylenetetramine (HTMA) to the solution, while keeping heating the solution, in various instances with equimolar amounts;
as illustrated in section a)-3 of FIG. 2, mixing the above solution while heating said solution in various instances at a slightly higher temperature comprised between 7° and 100° C., and this during a certain duration, i.e., of at least 60 minutes;
as illustrated in section a)-4 of FIG. 2, centrifugating the solution, while having stopped heating, i.e., at room temperature, so as to separate ZnO nanorods from the solution.

As illustrated in section a)-5 of FIG. 2, the centrifugated ZnO nanorods can be added to a solvent solution, like ethanol, optionally with CuCl₂, in preparation the further steps for growing the copper oxides on the ZnO nanorods.

The above method of synthetizing ZnO nanorods is as such known, notably from the patent application WO 2019/057786 A1 assigned to the applicant of the present application. Other methods can also be considered.

Section b) of FIG. 2 illustrates the growing of Cu₂(OH)₃Cl nanoplates on the above synthetized ZnO nanorods. It is to be understood that the ZnO nanorods do not necessarily need to be synthetized according to the above method as illustrated in section a) of FIG. 2.

As this is apparent in section b) of FIG. 2, a solution of ZnO nanorods and copper precursor such as CuCl₂ in a solution, being for instance water, is mixed with ammonia NH₄OH and sodium hydroxide NaOH so as to increase the pH of the resulting solution, e.g up to at least 12, in various instances at room temperature. This results in obtaining a precipitate with Cu(OH)₂ nanowires and the ZnO nanorods.

Thereafter, the solution is heated, in various instances between 6° and 80° C., and the pH of the solution is lowered by adding an acid, like chloride acid HCl, to a value of not more than 8 and more than 5, in various instances comprised between 6 and 7.5, where botallackite (Cu₂(OH)₃Cl) nanoplates are formed. Reducing the pH to a lower value would form clinoatacamite (Cu₂(OH)₃Cl) nanocrystals which is not desired.

The obtained ZnO nanorods with Cu₂(OH)₃Cl nanoplates are illustrated in section c) of FIG. 2. The solution can then be centrifugated and washed so as to separate the ZnO nanorods with Cu₂(OH)₃Cl nanoplates for being then applied onto a substrate for forming a gas sensor, a photocatalyst and a water splitter. In alternative, the ZnO nanorods with Cu₂(OH)₃Cl nanoplates can be stored in a solvent, like ethanol.

As stated here above in connection with FIG. 1, the ZnO nanorods with Cu₂(OH)₃Cl nanoplates are annealed for converting the Cu₂(OH)₃Cl nanoplates into CuO nanoplates. The annealing is done at a temperature comprised between 10° and 400° C. with a duration of at least one hour, in various instances at least two hours.

The conversion of the Cu₂(OH)₃Cl nanoplates into CuO nanoplates has been confirmed by high resolution X-ray photoelectron spectroscopy (XPS) of samples, the Cu 2p_{3/2} peak showing a substantially higher peak in the atom counts for the binding energy of 933.6 eV corresponding to CuO after annealing compared with before annealing. Cu₂(OH)₃Cl or Cu(OH)₂ are also present as confirmed by a peak for the binding energy of 934.8 eV, with a peak height reduced after annealing as compared with before annealing. As an examples, the mass ratio between Cu in CuO and Cu in copper hydroxide (Cu₂(OH)₃Cl or Cu(OH)₂) is evaluated by a fit of the XPS Cu 2p_{3/2} peak, and is found to be 0.5 before annealing, and 2.5 after 2 hours annealing at 400° C.

The conversion of the Cu₂(OH)₃Cl nanoplates into CuO nanoplates has also been confirmed by X-ray diffraction analysis (XRD) of samples, where the peaks at angles of 15.5 degrees, corresponding to botallackite Cu₂(OH)₃Cl, at angles of 16.7 degrees, corresponding to paratacamite Cu₂(OH)₃Cl, and at angles of 16.2 and 23.8 degrees, corresponding to Cu(OH)₂, are suppressed after annealing, and the peaks at angles of 35.5 and 38.9 degrees, corresponding to CuO, appear after annealing.

The conversion of the Cu₂(OH)₃Cl nanoplates into CuO nanoplates also changes the optical properties of the composite material. For instance, optical absorbance analysis of samples in the wavelength range from 250 nm to 1500 nm have been made and revealed that the annealed composite material showed a substantially higher absorbance in the visible range (from 300 to 800 nm) and also that the absorbance is higher for samples annealed at higher temperatures compared with those annealed at lower temperatures, these temperatures being comprised between 10° and 400° C.

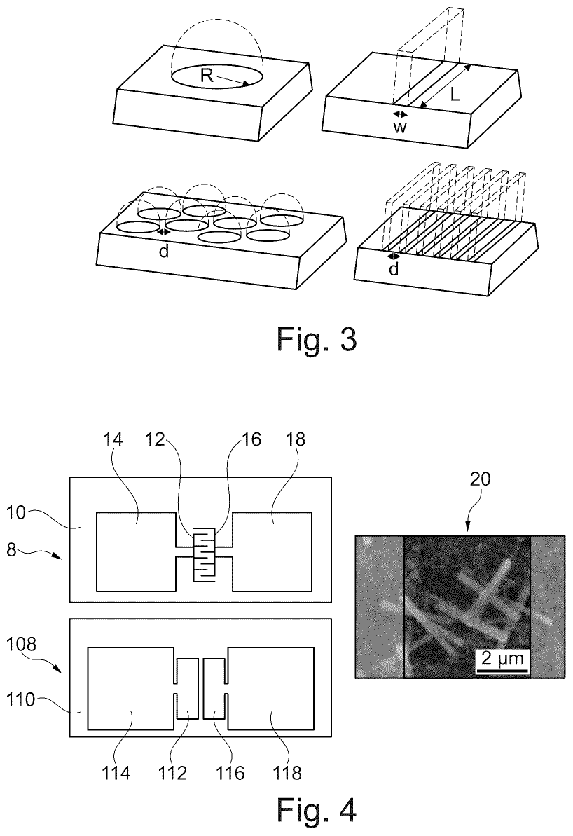

FIG. 3 illustrates the geometry of nanoparticles versus nanoplates.

The top left image shows a droplet or particle-shaped element attached to a support, the lower left image shows an array arrangement of the droplet or particle shaped elements. The top right image shows a plate-shaped element attached to a support and the lower right image shows an evenly distributed arrangement of the plate-shaped elements on the support.

For the droplet or particle-shaped element, we have

$$\frac{P}{A} = \frac{2}{R}$$

$${lim}_{R \to 0} \frac{P}{A} = ∞$$

$${lim}_{R \to 0} P = 0$$

where R is the radius of the droplet or particle-shaped element, P and A are the perimeter and surface on the support, respectively, of the droplet or particle-shaped element. It follows that droplet or particle-shaped element of a nano radius will show a surface that proportionally diminishes compared with the perimeter, i.e., the place that it occupies on the substrate.

If we consider the above-mentioned array of droplet or particle-shaped elements, we have

$$\frac{{P}_{tot}}{{A}_{tot}} = \frac{2 π R}{\frac{\sqrt{3}}{4} {( 2 R + d )}^{2}}$$

$${lim}_{R \to 0} \frac{{P}_{tot}}{{A}_{tot}} = 0$$

where R is the radius of the droplet or particle-shaped element, Pₜₒₜ and Aₜₒₜ are the total perimeter and total covered surface of the droplet or particle-shaped elements, and d is the distance between directly adjacent droplet or particle-shaped elements. It follows that an array of droplet or particle-shaped elements will show a total perimeter, i.e., a sum of the perimeters of all droplet or particle-shaped element of the array, being representative of the active surface that can be contacted by a gas, that will progressively become smaller than the total surface that the droplet or particle-shaped elements occupy on the substrate, as the radius R of the droplet or particle-shaped elements reaches a nanoscale.

For the plate-shaped element, we have

$$\frac{P}{A} = \frac{2}{w} + \frac{2}{L}$$

$${lim}_{R \to 0} \frac{P}{A} = ∞$$

$$P = 2 L + 2 w$$

$${lim}_{w \to 0} P = 2 L$$

where L is the length and w the width (in fact thickness) of the plate-shaped element, P and A are the perimeter and surface on the support, respectively, of the plate-shaped element. It follows that plate-shaped element of a nano width w will keep a perimeter proportional to its length L while the place that it occupies on the substrate diminishes.

If we consider the above-mentioned even distribution of plate-shaped elements, we have

$$\frac{{P}_{tot}}{{A}_{tot}} = \frac{2 L + 2 w}{( w + d ) L}$$

$${lim}_{w \to 0} \frac{{P}_{tot}}{{A}_{tot}} = \frac{2}{d}$$

where Pₜₒₜ and Aₜₒₜ are the total perimeter and total covered surface of the plate-shaped elements, and d is the distance between directly adjacent plate-shaped elements. It follows that reducing the width w to a nano scale maintains a fixed proportionality between the total perimeter, being representative of the of the active surface that can be contacted by a gas, with the total surface on the support that is occupied by the plate-shaped elements.

The above demonstrates the advantages of the above transversal arrangement of copper oxide nanoplates on the ZnO nanorods, with regard to optimizing the total contact surface for ambient gas for a given number and size of the nanorods. This thereby optimizes the sensitivity of the composite material.

The above-described ZnO nanorods show a length that can be comprised between 1 and 10 μm and a diameter comprised between 50 and 600 nm, resulting in a shape factor comprised between and 1.7 and 200. The above-described transversal nanoplates of copper oxide are distributed along the length of the ZnO nanorods with a density comprised between 10 and 100 per μm. The transversal nanoplates of copper oxide can show a thickness (corresponding to w in FIG. 3) comprised between 15 and 35 nm. The length (corresponding to L in FIG. 3) can be comprised between 800 and 2300 nm, and the height (perpendicular to w and L in FIG. 3) can be comprised between 300 and 630 nm. The above ranges are also disclosed, each, in isolation and each range is disclosed with its lower limit only and also with its upper limit only.

Also, the above-described transversal nanoplates of copper oxide are transversal to the ZnO nanorods, in various instances a normal parallel to a longitudinal axis of the ZnO nanorods or inclined relative to said longitudinal axis by not more than 45°, more in various instances perpendicular to the ZnO nanorods within a tolerance of ±15°, for example within a tolerance of ±10°.

Also, the above-described ZnO nanorods show a polygonal cross-section forming facets onto which the transversal nanoplates of copper oxide are attached.

FIG. 4 illustrates two embodiments of a gas sensor according to the invention.

The first embodiment is a gas sensor 8 comprising a substrate 10, a first electrode 12 and a first contact area electrically connected to the first electrode 12. Similarly, the gas sensor comprises a second electrode 16 and a second contact area electrically connected to the second electrode 12. As this is apparent the first and second electrodes 12 and 16 are interdigitated. The first and second electrodes 12 and 16, and the first and second contact areas 14 and 18 are formed by deposition of an electrically conductive material, i.e., copper based, on the substrate 10. The first and second electrodes 12 and 16 as well as directly adjacent areas of the substrate 10 are covered by a layer of ZnO nanorods with transversal nanoplates of CuO as detailed here above, forming a gas sensing layer 20.

The second embodiment is a gas sensor 108 similar to the gas sensor 8 of the first embodiment, where however the first and second electrodes 112 and 116 are not interdigitated. Similarly to the first embodiment, the first and second electrodes 112 and 116 as well as directly adjacent areas of the substrate 110 are covered by the gas sensing layer 20.

The gas sensors 8 and 108 can further comprise an electrical resistor dimension for bringing the substrate, the at least two electrodes and the gas sensing layer at a temperature of at least 100° C. and up to 250° C.

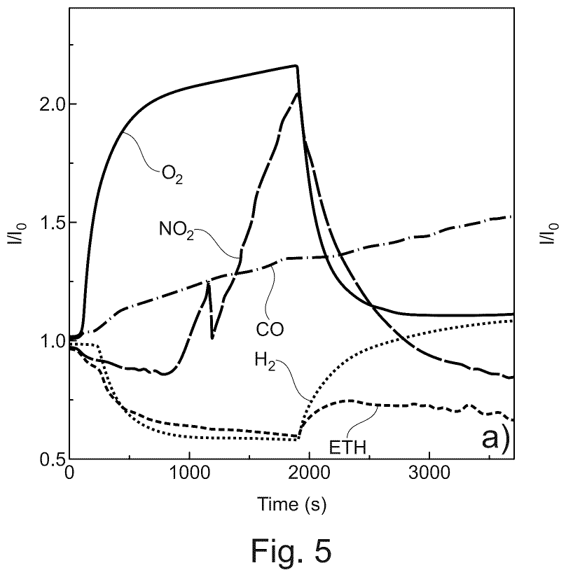

FIG. 5 is a graphic illustrating the electrical behavior of the gas sensor according to the invention in contact with various gases.

The above-described gas sensor has been tested in contact with various gases and its electrical behavior has been observed and reported in the graphic in FIG. 5. An electrical source, i.e., a voltage source, has been electrically connected to the contact areas of the gas sensor, while contacting its gas sensing layer with N₂ and maintaining the gas sensor at a controlled temperature of 150° C., resulting in a stabilized nominal current I₀ flowing through the gas sensor. The gas sensor has then been brought into contact different types of gas as will be explained here after, and the current/has been measured. To that end, the ambient atmosphere of the gas sensor is saturated successively with each of the different types at given concentrations.

In practice, the gas sensor is located in a generally closed chamber that is fed with either pure N₂ and thereafter with the gas to be tested, in the case the gas to be tested is O₂, or dry air and thereafter with the gas to be tested, in the case the gas to be tested is H₂, CO, NO₂ or ethanol. The chamber comprises at least one exhaust so as to allow the gas to be tested to flow, i.e., to be renewed in the chamber, i.e., about every 30 seconds.

The gas sensor has been brought into contact with O₂ at a concentration of 20.9% mixed N₂ as a carrier gas, during 1800 seconds (i.e., 30 minutes). We can observe a rapid increase in the current response, where I/I₀ reaches 2 after about 600-700 seconds. Thereafter the increase in the current response is less steep. After applying the above gas O₂ containing gas, for instance during 1800 seconds, pure N₂ is applied as before applying said O₂ containing gas. We can see a rapid decrease in the current response.

The gas sensor has also been brought into contact with H₂ at 2% in N₂, also during 1800 seconds, similarly to the above 02 and N₂ gas mixture. We can observe a current response that is opposed to the one with O₂, i.e., where the current diminishes instead of increasing. The current response I/I₀ reaches 0.6 after about 750 seconds, which is comparable with the above current response with O₂.

The gas sensor has also been brought into contact with CO at 20 ppm in air, also during 1800 seconds, similarly to the above 02-N₂ and H₂—N₂ gas mixtures. The response observed for CO is much different than that observed for H₂, even though both are reducing gasses.

The gas sensor has also been brought into contact with NO₂ at 8.26 ppm in air, also during 1800 seconds, similarly to the above gas mixtures. We can observe a substantial increase of the current response. The delayed response observed with this gas is due to an experimental artifact due to the large dilution of this specific gas. After stopping application of the above gas mixture, a rapid decrease of the current response can be observed.

The gas sensor has also been brought into contact with ethanol (ETH) at 100 ppm in air, also during 1800 seconds, similarly to the above gas mixtures. We can observe a current response that is similar to the one of H₂ in N₂, i.e., a decrease in the current response, whereas after application of the above test gas mixture, the recovery current behavior is quite different from the one with H₂ in N₂.

In summary, we observe in FIG. 5 a good sensitivity and selectivity to the tested gases, thereby showing the suitability and ability of the composite material of the invention and corresponding sensor for gas sensing applications, in particular for the above tested gases.

1.-18. (canceled)

19. A gas sensor, said gas sensor comprising:
a substrate;
at least two electrodes deposited on the substrate;
a gas sensing layer comprising ZnO nanorods deposited on the at least two electrodes and on the substrate between the at least two electrodes, the gas sensing layer showing an electrical resistivity that varies when contacted by the gas;
wherein the ZnO nanorods are provided with transversal nanoplates of oxidized copper so as to confer sensitivity of the gas sensing layer to the gas being at least one of O₂, H₂, CO, ethanol and NO₂.

20. The gas sensor of claim 19, wherein the ZnO nanorods have a length comprised between 1 and 10 μm.

21. The gas sensor of one claim 19, wherein the ZnO nanorods have a diameter comprised between 50 and 600 nm.

22. The gas sensor of claim 19, wherein the ZnO nanorods extend along a longitudinal axis and the transversal oxidized copper nanoplates are perpendicular to the longitudinal axis within a tolerance of ±15°.

23. The gas sensor of claim 19, wherein the ZnO nanorods provided with transversal nanoplates of oxidized copper have a number of the transversal nanoplates of oxidized copper that is comprised between 10 and 100 per μm of length of ZnO nanorod.

24. A metal oxide material, said metal oxide material comprising:
ZnO nanorods; and
oxidized copper particles attached to the ZnO nanorods;
wherein the oxidized copper particles are nanoplates extending transversally to the ZnO nanorods, the transversal oxidized copper nanoplates having a normal parallel to a longitudinal axis of the ZnO nanorods or inclined relative to the longitudinal axis by not more than 45°.

25. The metal oxide material of claim 24, wherein the ZnO nanorods have a length comprised between 1 and 10 μm.

26. The metal oxide material of claim 24, wherein the ZnO nanorods have a diameter comprised between 50 and 600 nm.

27. The metal oxide material of claim 24, wherein the transversal oxidized copper nanoplates are perpendicular to the longitudinal axis of the ZnO nanorods within a tolerance of ±15°.

28. The metal oxide material of claim 24, wherein the ZnO nanorods provided with transversal nanoplates of oxidized copper have a number of the transversal nanoplates of oxidized copper that is comprised between 10 and 100 per μm of length of ZnO nanorod.

29. A method of synthetizing ZnO nanorods provided with Cu-based nanoplates, said method comprising the following successive steps:
(a) preparing ZnO nanorods;
(b) mixing the ZnO nanorods with a Cu precursor in a solution;
(c) increasing the pH of the solution, so as to form Cu(OH)₂ nanowires in the solution;
(d) lowering the pH of the solution, so as to form Cu₂(OH)₃Cl nanoplates on the ZnO nanorods.

30. The method of claim 29, comprising the further step:
(e) annealing the ZnO nanorods provided with the Cu₂(OH)₃Cl nanoplates so as to transform at least partially the Cu₂(OH)₃Cl nanoplates into CuO nanoplates.

31. The method of claim 30, wherein annealing at step (e) is achieved at a temperature of at least 100° C. during at least one hour.

32. The method of claim 30, wherein annealing at step (e) is achieved at a temperature of at least 200° C. during at least one hour.

33. The method of claim 29, wherein step (c) comprises adding NaOH so as to increase the pH to at least 12.

34. The method of claim 29, wherein step (d) comprises adding HCl so as to lower the pH to a value comprised between 6 and 8.
